(19) United States
(12) Patent Application Publication
(10) Pub. No.: US 2015/0247848 A1
(43) Pub. Date: Sep. 3, 2015

(54) MATERIAL AND METHODS FOR DIAGNOSING AND TREATING KAWASAKI DISEASE AND KLS

(71) Applicant: Indiana University Research & Technology Corporation, Indianapolis, IN (US)

(72) Inventor: Raymond M. Johnson, Indianapolis, IN (US)

(21) Appl. No.: 14/427,576

(22) PCT Filed: Sep. 12, 2013
(86) PCT No.: PCT/US2013/059529

§ 371 Date: Mar. 11, 2015

Related U.S. Application Data
(60) Provisional application No. 61/700,105, filed on Sep. 12, 2012.

(51) Int. Cl. G01N 33/564; C07K 16/28; C07K 16/24

(52) U.S. Cl. CPC G01N 33/564; C07K 16/24; C07K 16/2866; C07K 2317/76; G01N 2333/5412; G01N 2333/5437; G01N 2333/7151; G01N 2333/523; G01N 2333/522

(57) ABSTRACT

Two patients diagnosed with KLS were treated. One patient had severe KLS that progressed to the equivalent of pediatric Kawasaki Disease Shock E Syndrome (KDSS). The second patient had a typical KLS presentation and clinical course. Cytokines and chemokines provide inflammatory signatures in the serum that reflect the polarity of the immune response and the affected cell types. Multiplex ELISA technology was used to define the cytokine milieu in the serum of the two adult IIIV patients with KLS during the acute and convalescent phases. Those sera were compared with sera from asymptomatic HIV subjects and a normal serum control. Those comparisons suggest that HIV KLS is a dysfunctional Th2 response to an unknown inciting agent in the vascular wall, and that a multiplex ELISA or similar technology based a limited combination of KLS/KD pathogenesis-related cytokines (IL-6, IL-13, sTNFRII) and endothelial/smooth muscle chemokines (CCL1, CCL2, CxCL11 may provide an objective tool for diagnosing KLS and Kawasaki Disease. Because KD and HIV KLS are the only known “Th2” vasculitidies that spare the lungs (unique clinical presentation) and include plasma cell infiltration of the vascular wall as a prominent histopathologic feature (unique pathophysiology), a diagnostic test based on combinations of the above analytes will be highly specific and therefore clinically useful.

### PRIORITY CLAIM

This Application claims the benefit of U.S. Provisional Patent Application No. 61/700,105 filed on Sep. 12, 2012. This provisional application is incorporated herein by reference in its entirety.

### FIELD OF THE INVENTION

Aspect of the invention related to diagnosing, treating, for studying and treating HIV Kawasaki-like syndrome (KLS) and Kawasaki disease.

### BRIEF DESCRIPTION OF THE FIGURES

FIG. 1 Photographs of patient number 1, from top to bottom panels showing rash, changes of the oropharynx, non-exudative conjunctivitis, and erythema with mild swelling of hands.

FIG. 2A Graphs, panels from top to bottom levels of: IFN-γ (pg/ml); TNF-α (pg/ml); and IL-1ra (pg/ml) isolated from KLS patient number 1 (black squares; severe KLS) and patient number 2 (gray circles; typical KLS).

FIG. 2B Graphs, panels from top to bottom levels of: CxCL10 (pg/ml); IL-10 (pg/ml); and OPG (pg/ml) isolated from KLS patient number 1 (black squares; severe KLS) and patient number 2 (gray circles; typical KLS).

FIG. 3A Graph of levels of IFN-α (pg/ml) isolated from same patients and controls as shown in FIG. 2A.

FIG. 3B Graph of levels of M-CSF (pg/ml) isolated from same patients and controls as shown in FIG. 2A.

FIG. 4A Graphs, panels from top to bottom levels of: IL-6 (pg/ml); IL-13 (pg/ml); and CCL2 (pg/ml) isolated from same patients and controls as shown in FIG. 2A.

FIG. 4B Graphs, panels from top to bottom levels of: sTNFRII (pg/ml); CCL1 (pg/ml); and CxCL11 (pg/ml) isolated from same patients and controls as shown in FIG. 2A.

FIG. 5 Diagram illustrating the effect of reduced CD4 T-cell counts on an adult patient's ability to respond to Kawasaki disease.

### BACKGROUND AND SUMMARY

Adult Kawasaki-like Syndrome (KLS) seen in HIV+ individuals is a dramatic vasculitis with fevers, conjunctivitis, rash, changes of the oropharynx, and erythematous/painful hands and feet, usually without the sentinel lymph node more commonly seen in pediatric Kawasaki Disease. The adult and pediatric syndromes share an unusual histopathology of IgA+ plasma cells within inflamed arterial walls. The distinctive clinical presentation and pathognomonic histopathology suggest a shared inciting etiology and atypical inflammatory response in HIV KLS and KD patients.

Pediatric Kawasaki Disease (KD) is diagnosed clinically based on a constellation of signs and physical exam findings including a fever ≧5 days plus at least four of the following five physical findings: non-exudative conjunctivitis, rash, changes of the oropharynx, erythema and/or painful swelling of the hands and feet, and a sentinel lymph node (>1 cm and tender). Kawasaki Disease is typically seen in children >6 months<5 years of age. Epidemiology of the disease includes periodic outbreaks suggestive of a ubiquitous infectious etiology, with an atypical presentation in a subset of individuals who come to medical attention as having Kawasaki Disease. KD inflammation has predilection for coronary arteries, with serious sequelae including development of coronary artery aneurysms and residual long term risk for increased cardiovascular morbidity and mortality. In children, therapy with IVIG reduces morbidity and mortality from Kawasaki disease by decreasing the proportion of patients with coronary artery aneurysm formation from 20% to 3%-4%. Diagnosis of KD by clinical criteria is problematic as children presenting with partial syndromes (fever plus 3 or fewer physical findings) may not receive IVIG and aspirin therapy, but can develop coronary artery aneurysms and sudden death.

Kawasaki Disease is extremely rare in adults. In the 1980s cases of an adult febrile syndrome that resembled pediatric Kawasaki Disease came to medical attention. The majority of the adults affected were noted to be HIV⁺, most with advanced disease and low CD4 counts. HIV⁺ individuals with Kawasaki-like syndrome (KLS), also known as Kawasaki Disease-like syndrome (KDLS), commonly experience a gastrointestinal disturbance with diarrhea and/or abdominal pain followed within several weeks by development of the protean manifestations of the syndrome including fevers, conjunctivitis, rash, changes of the oropharynx (strawberry tongue, fissures, cheilitis), painful erythema+/−swelling of the hands and feet, usually without a sentinel lymph node. HIV KLS patients respond to IVIG and aspirin, and therefore have been treated similar to pediatric KD patients (reviewed in (1)). Based on very limited evaluations, no HIV KLS patient has been documented to have developed coronary artery aneurysms; however cases of coronary artery aneurysm formation have been documented in HIV negative adults with KD.

Kawasaki Disease is the only vasculitis syndrome that includes infiltration of vessel walls with IgA⁺ plasma cells. Henoch-Scholein Purpura is an IgA-associated vasculitis that includes IgA deposition without infiltration by IgA+ plasma cells. The coronary arteries of children with fatal Kawasaki disease show mixed inflammatory cells with a prominent population of IgA⁺ plasma cells. KD autopsies have shown that the IgA plasma cell infiltration includes multiple vascular and non-vascular tissues throughout the body (2). The IgA plasma cell finding has led to the hypothesis that Kawasaki Disease in an infectious agent that originally invades through the respiratory tract or gut, as IgA plasma cell ontogeny and immunoglobulin class switching occur predominantly in mucosal tissues. A conjunctival biopsy of an HIV⁺ patient with KLS showed IgA⁻ plasma cells infiltrating arterial walls. That critical finding linked HIV KLS with pediatric Kawasaki Disease at the level of histopathology (3). The same study showed markedly elevated sTNFRII levels in serum during the acute phase of HIV KLS, suggesting a major role for TNFα in KLS pathophysiology as serum levels of soluble TNF receptors are regulated by TNFα.

Understanding the pathophysiology of HIV KLS may improve therapeutic interventions and contribute toward development of a practicable diagnostic test that would at least partially alleviate current dependence on a constellation of clinical signs and physical findings combined with extensive diagnostic testing to rule out other etiologies. Development of a reliable KLS/KD diagnostic test is important as individuals who present with incomplete Kawasaki syndromes may not receive IVIG therapy and can go on to develop the vascular sequelae. A reliable diagnostic test would also shorten hospitalizations and curtail an expensive diagnostic evaluation to rule out other etiologies. Fortuitous presentation of two acute cases of HIV KLS, one severe and one typical, allowed us to investigate KLS inflammation in the acute and convalescent phase. Interesting results of those investigations are presented here.

Some aspects of the invention include diagnostic methods based on the unique pathophysiology of KLS/KD, comprising the steps of; quantifying human proteins in a serum sample from a patient, the proteins selected from of the group consisting of: IL-6, IL-13, sTNFRII, CCL1 (I-309), CCL2 (MCP-1), and CxCL11 (I-TAC), wherein the patient exhibits at least one symptom of Kawasaki disease or KLS; measuring the level of plasma cell recruiting chemokine CxCL11 and/or “Th2” biomarkers IL-13 & CCL1 (singly or in combination) and/or vascular inflammation marker CCL2 isolated from the sample; and tagging the sample as being from a patient positive or preliminarily positive for KD or HIV KLS if the level of plasma cell recruiting chemokine CxCL11 and/or “Th2” biomarkers IL-13 & CCL1 (singly or in combination) and/or vascular inflammation marker CCL2 isolated from the sample is 2 or more Standard Deviations higher than are the levels of these proteins in serum of a similar patient, wherein the similar patient is asymptomatic for KLS and KD.

Some embodiments for diagnosing KD or HIV KLS, further including the step of: validating that the patient is positive for KD or HIV KLS by determining if the levels of sTNRII and/or IL-6 isolated from the patients' sample is 2 or more Standard Deviations higher than the levels of these proteins isolated from the serum of a similar patient, wherein the similar patient is asymptomatic for KLS and KD. In some embodiments the isolations steps includes the use of a Multiplex ELISA. In some embodiments the isolation step includes the use of a strip testing technology. In still other embodiments the isolation step includes the use of mass spectroscopy identification technique.

Some aspects of the invention include a method of treating a patent for KD or HIV KLS treatment method, comprising the steps of: administering a therapeutically effective dose of at least one compound that interferes with the interactions of at least one receptor ligand pairs selected from the group consisting of: CxCL11-CCR3, CCL1-CCR8, and CCL2-CCR2 wherein the patient is afflicted with KS or HIV KLS. In some embodiments the compound is at least one antibody that binds to at least one of protein selected from the group consisting of: CxCL11, CCRb3, CCL1, CCR8, CCL2, and CCR2.

A first embodiment includes, methods for treating a patient based on the unique pathophysiology of KLS/KD, comprising the steps of: quantifying at least one human protein in a serum sample from a patient, wherein the protein is selected from the group consisting of: IL-6, IL-13, sTNFRII, CCL1 (I-309), CCL2 (MCP-1), and CxCL11 (I-TAC), wherein the patient exhibits at least one symptom of Kawasaki disease or KLS; measuring the level of “plasma cell recruiting” chemokine CxCL11 and/or “Th2” biomarkers IL-13 & CCL1 (singly or in combination) and/or “vascular inflammation marker” CCL2 in the serum sample; and tagging the sample as being from a patient positive or preliminarily positive for KD or HIV KLS if the level of “plasma cell recruiting” chemokine CxCL11 and/or “Th2” biomarkers IL-13 & CCL1 (singly or in combination) and/or “vascular inflammation marker” CCL2 isolated from the sample is 2 or more Standard Deviations higher than are the levels of these proteins in the serum of a similar patient, wherein the similar patient is asymptomatic for KLS and KD.

A second embodiment includes methods according to the first embodiment, further including the step of: validating that the patient is positive for KD or HIV KLS by confirming that the levels of sTNRII and/or IL-6 quantified in the patients' sample is 2 or more Standard Deviations higher than the levels of these proteins isolated from the serum of a similar patient, wherein the similar patient is asymptomatic for KLS and KD.

A third embodiment includes methods according to the first and the second embodiments, wherein the quantifying and/or measuring steps include the use of a Multiplex ELISA.

A fourth embodiment includes the methods according to first and the second embodiments, wherein the quantifying and/or measuring steps include the use of a strip testing technology.

A fifth embodiment includes the methods according to first and the second embodiments wherein the quantifying and/or measuring steps include the use of a mass spectroscopy identification technique.

A sixth embodiment includes treatment methods, comprising the steps of: administering a therapeutically effective dose of at least one compound that interferes with the interactions of at least one receptor ligand pair selected from the group consisting of: CxCL11-CCR3, CCL1-CCR8, and CCL2-CCR2 wherein the patient is afflicted with KS or HIV KLS.

A seventh embodiment includes treatment methods according to the sixth embodiment, wherein the compound is at least one antibody that binds to at least one protein selected from the group consisting of: CxCL11, CCR3, CCL1, CCR8, CCL2, and CCR2.

An eighth embodiment includes systems for treating a patient, comprising: a human serum sampling handling device; at least one protein probe that selectively binds at least protein selected from the group consisting of: IL-6, IL-13, sTNFRII, CCL1 (I-309), CCL2 (MCP-1), and CxCL11 (I-TAC); a detector that monitors the at least one probe and records a signal from the probe that is proportional to the level of at least protein in the serum sample, where the at least one protein is selected from the group consisting of; IL-6, IL-13, sTNFRII, CCL1 (I-309), CCL2 (MCP-1), and CxCL11 (I-TAC) and wherein the detector produces a signal that is proportional to the level of the at least one protein selected from the group consisting of; IL-6, IL-13, sTNFRII, CCL1 (I-309), CCL2 (MCP-1), and CxCL11 (I-TAC); a central processing unit, wherein the central processing unit is configured to receive the signal from the detector and to access a matrix of values for the levels of at least one protein selected from the group consigning of IL-6, IL-13, sTNFRII, CCL1 (I-309), CCL2 (MCP-1), and CxCL11 (I-TAC), determined in patients that are both diagnosed with and diagnosed without Kawasaki Disease or KLS, wherein the central processing unit compares the levels of the at least one protein quantified in the serum sample and in the matrix and produces an output; and a user interface that receives the output from the central processing unit and displays a readable image of the comparison of the levels of protein in the serum sample and the values in the matrix.

A ninth embodiment includes systems according to the eighth embodiment, wherein the sample handling device is selected from the group consisting of: single well plates, multi-well plates, tubes, vials, syringe bodies, and aspirators.

A tenth embodiment includes systems according to the eighth and ninth embodiments, wherein the protein probe includes at least one antibody that has been raised to at least of the proteins selected from the group consisting of: IL-6, IL-13, sTNFRII, CCL1 (I-309), CCL2 (MCP-1), and CxCL11 (I-TAC).

An eleventh embodiment includes systems from the eighth through the tenth embodiments, wherein the protein probe comprises a Multiplex ELISA.

A twelfth embodiment includes systems from the eighth through the eleventh embodiments, wherein the detector records a change in at least one signal generated by the interaction between proteins in the serum sample the protein probe, wherein the signal is selected from the group consisting of: luminescence, fluorescence, conductivity, chemiluminescence, and radiation.

A thirteenth embodiment includes systems from the eighth through the twelfth embodiments, wherein the central processing unit is a digital computer.

A fourteenth embodiment includes systems from the eighth through the thirteenth embodiments, wherein the user interface is selected from the group consisting of: a monitor and a printer.

A fifteenth embodiment includes systems from the eighth through the fourteenth embodiments, wherein the central processing unit is configured to produce an output that includes flagging a difference in the level of at least one protein selected from the group consisting of; IL-6, IL-13, sTNFRII, CCL1 (I-309), CCL2 (MCP-1), and CxCL11 (I-TAC), of at least 1 standard deviation measured between said proteins in the serum sample and the values for said proteins stored in the matrix for individuals that have not been diagnosed with Kawaski disease or KLS.

A sixteenth embodiment includes systems from the eighth through the fifteenth embodiments, wherein the central processing unit is configured to produce an output that includes flagging a difference in the level of at least one protein selected from the group consisting of: IL-6, IL-13, sTNFRII, CCL1 (I-309), CCL2 (MCP-1), and CxCL11 (I-TAC), of at least 2 standard deviations measured between said proteins in the serum sample and the values for said proteins stored in the matrix for individuals that have not been diagnosed with Kawaski disease or KLS.

A seventeenth embodiment includes systems from the eighth through the sixteenth embodiments, wherein the central processing unit is further configured to receive at least one additional input, wherein the at least one additional input includes the presence of a least one gross physical manifestation of Kawaski disease or KLS in the patient from which the serum sample was obtained.

An eighteenth embodiment includes systems from the eighth through the seventeenth embodiments, wherein the central processing unit is further configured to produce an output that notes the existence of the existence of the at least one gross physical manifestation of Kawaski disease or KLS and transmits the note to the user interface.

### DESCRIPTION

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

As used herein, unless explicitly stated otherwise or clearly implied otherwise, the term ‘about’ refers to a range of values plus or minus 10 percent, e.g., about 1.0 encompasses values from 0.9 to 1.1.

As used herein, unless explicitly stated otherwise or clearly implied otherwise, the term ‘method of treating’ includes such as providing therapeutic levels of specific compounds to a patient, diagnosing disease or abnormality in a patient, and/or providing medical advice to a patient.

As used herein, unless explicitly stated otherwise or clearly implied otherwise, the terms ‘therapeutically effective dose,’‘therapeutically effective amounts,’ and the like, refers to a portion of a compound that has a net positive effect on the health and well-being of a human or other animal. Therapeutic effects may include an improvement in longevity, quality of life and the like these effects also may also include a reduced susceptibility to developing disease or deteriorating health or well-being. The effects may be immediate realized after a single dose and/or treatment or they may be cumulative realized after a series of doses and/or treatments.

### EXAMPLE 1

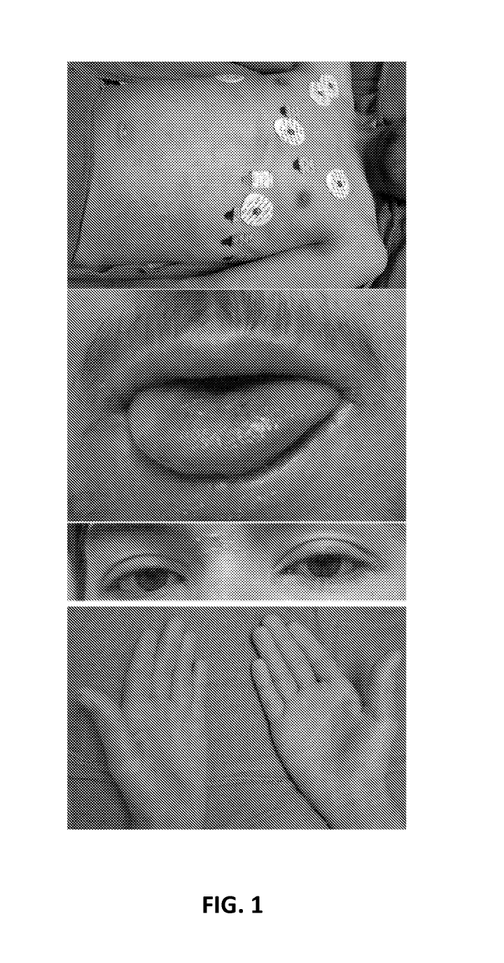

Patient 1 was a 23 year old South American male with a history of IVDA/cocaine/marijuana use residing in the US for one year who was admitted from the emergency room with two days of fevers, rash, strawberry tongue, and conjunctivitis preceded by several months of low grade diarrhea, progressing to include NN and abdominal pain during the week preceding admission. On admission his main complaints were fevers, rash and weakness. His admitting vitals were: Temp 101.9° F., HR 142, BP 68/47, RR 16, pulse oximetry 100% on RA. His basic labs showed: WBC 5.0, Hct 41, Plt 83, Na 128, Bicarb 20, K 2.5, Cl 90, BUN 42, Scr 3.9, AST 167, ALT 71; CXR was unremarkable. His admitting diagnoses were fever/diarrhea/dehydration, and he was treated empirically with vancomycin, ceftriaxone and clindamycin for presumed group A strep or mild toxic shock. Patient's BP, HR, electrolytes and Scr normalized with hydration and associated hemoconcentration resolved revealing an underlying anemia and hypoalbuminemia, but the fevers persisted in spite of antibiotic therapy. He was screened for HIV and viral hepatitis and found to be HIV⁺ with an absolute CD4 count of 3 and viral load of 180,000 copies per ml, and to be chronically infected with Hepatitis C virus (HCV). Over the ensuing week the patient remained normotensive and febrile while undergoing an extensive diagnostic evaluation. Toward the end of the 1st week of hospitalization he was clinically deteriorating with worsening fevers up to 103.9° F. and episodes of hypotension responsive to fluid resuscitation. There were multiple adjustments to his antibiotic therapy during the first week including courses of first & fourth generation cephalosporins, vancomycin, trimethoprim/sulfamethoxazole, and addition of amphotericin B without improvement of his clinical status. On the eight day of hospitalization he was transferred to the ICU, and semi-electively intubated for ongoing fevers, respiratory rate of 40, and impending vascular collapse. Photos of patient's physical findings at time of ICU transfer are shown in FIG. 1. In the ICU his CXR showed increased interstitial markings and he had a bronchoscopy with BAL with negative stains for PCP, yeasts, molds, viral, or bacterial pathogens; final cultures grew a few Candida albicans. On day 9 of hospitalization, patient had ongoing fevers and required pressor therapy; the available diagnostic evaluation for infectious agents was entirely negative. The complexity of the patient's case, severity of his illness, and deteriorating clinical course led to a broad informal consultation among the infectious disease faculty that resulted in consideration of KLS. The patient's exam on the 9th day of hospitalization included conjunctivitis, erythematous swollen hands and feet, a macular rash, and an endotracheal tube obstructing the visual exam of an obviously macerated oropharynx. He was sedated, easily oxygenated with an FiO₂ of 0.40, without other significant physical findings except tachycardia. He remained febrile in spite of empiric hydrocortisone, vancomycin, meropenum, doxycycline, amphotericin B, and metronidazole. Based on fevers, conjunctivitis, changes of oropharynx and extremities and a rash, the possibility of KLS and IVIG and aspirin therapy were discussed with the intensive care unit staff. Later that day the patient became severely hypotensive requiring maximal fluid resuscitation and maximal pressor support (norepinephrine and phenylephrine). An echocardiogram showed hyperdynamic ventricles with preserved function. Without other changes in his medications the patient was treated for HIV KLS with 2 gm/kg of IVIG. Aspirin therapy was not initiated due to a platelet count of 33; instead methylprednisolone was added to hydrocortisone.

The patient's hypotension rapidly responded to the IVIG infusion plus steroids, including coming off pressor support within 24 h of completing IVIG and resolution of fevers. He made rapid and steady improvement, however four days after IVIG therapy he had recurrent fevers to 101.5° F. while still on methylprednisolone. On hospital day 17, eight days after IVIG#1, the patient was having fevers to 103.6° F. on prednisone plus vanc/cefepime, but he was otherwise doing well. On hospital day 18, nine days after IVIG#1, antibiotics were discontinued and the patient received a second dose of IVIG (IVIG#2). Patient's fevers improved slightly to <102° F. after IVIG#2, but did not resolve during the following week. Steroids were tapered off and on day 6, status post IVIG#2 (hospital day 24) the patient was having high grade fevers (103.3° F.) while on scheduled acetaminophen and ibuprofen therapies. The patient's platelet count was now 285 and the possibility of aspirin (ASA) therapy was revisited. ASA 650 mg po qid was started and the patient defervesced within 24 h. The inpatient medical service was uncomfortable with the high dose ASA therapy and the patient was switched to 50 mg prednisone+165 mg ASA daily. He had fine desquamation of the skin of the distal fingers and toes, and around the eyes. He sloughed all the epithelium from his tongue. Remarkably he suffered no other residual end organ damage and was discharged to home on day 11 s/p IVIG#2 (hospital day 29) on a regimen of 165 mg ASA daily, a 2 week prednisone taper, and bactrim/azithromycin prophylaxis for PCP/MAC. A summary of his diagnostic evaluation is included in Table 1.

The patient had follow-up outpatient clinic visits one, five, and thirteen weeks after hospital discharge. He started protease inhibitor-based cART with eventual immune reconstitution (CD4 293; HIV vl<50 copies/ml two years later). He had a negative cardiac stress thallium study 6 months after discharge from the hospital and went off aspirin therapy. With the patient's permission, serum collected immediately prior to the first dose of IVIG during his hospitalization and convalescent serum collected at a clinic visit 13 weeks post discharge were stored at −80° C. for possible future investigation of KLS. The patient did not have a KLS recurrence during the next 5 years, and was lost to follow up when he returned to his native country.

### EXAMPLE 2

Patient 2 was a 31 year old African American male recently diagnosed with HIV, CD4 count of 19, an HIV viral load of 139,000 copies per ml, with a pre-existing idiopathic eosinophilia who was admitted from the ER with ten days of fever (102.7° F.), myalgias, mild abdominal pain with occasional diarrhea, headaches, and painful swelling of the hands and feet beginning roughly coincident with initiation of 1st cART regimen (tenofovir/emtricitabine/ritonavir-lopinavir). He was not on PCP prophylaxis as the CD4 count was pending at time of previous clinic visit, and he had a questionable history of a sulfa drug allergy. His past medical history was remarkable for secondary syphilis 1 year earlier treated with IM benzathine PCN. His vital signs on admission were: Temp 103.7° F., HR 90, BP 110/63, RR 16. His physical exam was remarkable for non-exudative conjunctivitis, mild thrush, non-tender cervical lymphadenopathy all <1 cm, impressive painful swelling of the hands and feet, an erythematous rash somewhat difficult to appreciate due to dark skin pigment. Basic admission labs were unremarkable: WBC 10.0, HCT 35, Plts 382, Alk Phos 91, ALT 32, Alb 3.6, and Scr 1.1. The admitting medicine team did not initiate antibiotic therapy; the patient's cART was held, an infection workup was initiated, and ophthalmology, infectious diseases and rheumatology consults were requested. The working diagnoses were drug reaction versus Chlamydia-associated reactive arthritis. The ID consultant evaluating the patient was suspicious that the patient had KLS, and pursued additional input within the Infectious Diseases division. On day 5 of the patient's hospitalization he remained febrile to 103° F., available diagnostic testing was negative (including Chlamydia urine ligase chain reaction), and on the basis of clinical criteria he received 2 gm/kg IVIG plus ASA 325 mg po qid. After completing the IVIG infusion he had no further fevers, felt remarkably better, and was discharged from the hospital the following day, resuming prior cART plus ASA 325 mg bid, azithromycin MAC prophylaxis, and fluconazole for thrush. A G6PD was pending at discharge with plans to initiate dapsone PCP prophylaxis during outpatient follow up. His diagnostic evaluation is included in Table 1.

He returned to the ID clinic 2 and 5 weeks after discharge for follow up care, and was doing well on both visits. He had significant periungual desquamation of the hands on the 2 week follow up visit. With the patient's permission, serum collected immediately prior to the first dose of IVIG during his hospitalization and convalescent serum collected at his clinic visit 5 weeks post discharge were stored at −80° C. for possible future investigation of KLS.

### Multiplex ELISA

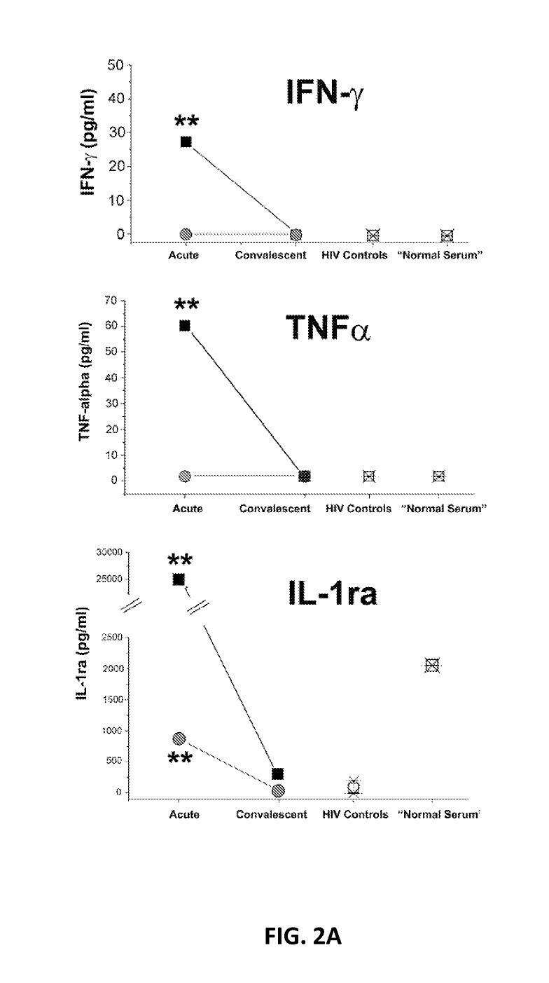

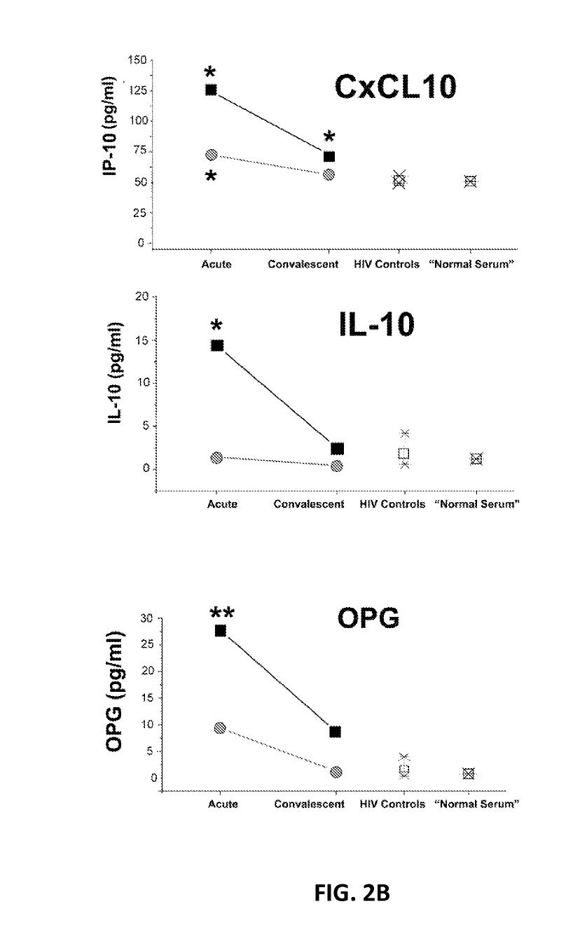

Advances in multiplex ELISA technology offered new opportunities to investigate KLS using the acute and convalescent serum stored at −80° C. Multiplex ELISA makes it possible to determine levels of multiple analytes using a small volume of patient serum. A protocol was approved by the Indiana University Institutional Review Board to do a case control study with the frozen KLS patient sera, leftover pretreatment serum from an unrelated endovascular function study of HIV subjects with CD4 counts >350 not taking cART, and serum from an HIV negative control subject. The single HIV negative normal serum was included to preliminarily identify analytes that may be abnormally low in HIV⁺ individuals (e.g. IL-1ra; FIG. 2A). A custom multiplex ELISA was designed incorporating the analytes detailed in Table 2. The analytes were a collection of chemokines, cytokines, and biomarkers representing cytokine polarization patterns (Th1 versus Th2), tissue and cell type specific inflammation, and/or prior association with atherosclerosis or KD/KLS. It was not possible within financial constraints to derive normative values for the analytes in the panel. Existing non-standardized serum levels of each analyte in the panel from published KD/KLS clinical studies as available are annotated in Table 3. The KLS and control sera were adjusted to 0.5% NP40 per the protocol of a commercial provider of multiplex ELISA testing (Pierce Biotechnology Searchlight [now Aushon Biosystems]; Woburn, Mass.). The commercial lab performed multiplex ELISA testing on samples labeled 1-8 without additional descriptors (blinded). Samples 1 & 2 were paired acute/convalescent sera from patient 1; samples 3 & 4 were paired acute/convalescent sera from patient 2; samples 5-7 were asymptomatic male HIV control subjects CD4 >350; sample 8 was an HIV-negative asymptomatic female control subject (entire data set provided as supplemental table 1). To complete the data set a standard ELISA was performed in house to quantify IFNy in samples 1-8 according to the manufacturer's protocol (human IFN-γ ELISA kit; Pierce-Endogen; Rockford, Ill.).

The data was analyzed as individual comparisons of patient 1 and patient 2 acute and convalescent values to the combined analyte data from the three asymptomatic HIV⁺ control subjects with a Student's T-test to determine statistical significance within this small clinical cohort. The serum results from the HIV negative control “normal serum” were not included in the statistical analysis. For within-the-study statistical comparisons, p values of <0.05 are considered statistically significant and are indicated by *; p values <0.01 are indicated by **. Correction for multiple comparisons were not performed because all analytes in the panel had previously been reported to be elevated in KLS or KD, or were logically linked with KLS pathogenesis (CCL1, IL-13, and CxCL11).

The results of the multiplex ELISA testing can be grouped into three functional categories. In the first category are analytes that were not elevated in the KLS patients compared to asymptomatic HIV⁺ control subjects. This category includes IL-17 and CCL5 (Rantes) (data not shown); and IL-1β that was below the limit of detection (<0.4 pg/ml) in the two KLS patients, the three HIV control subjects, and the HIV negative serum.

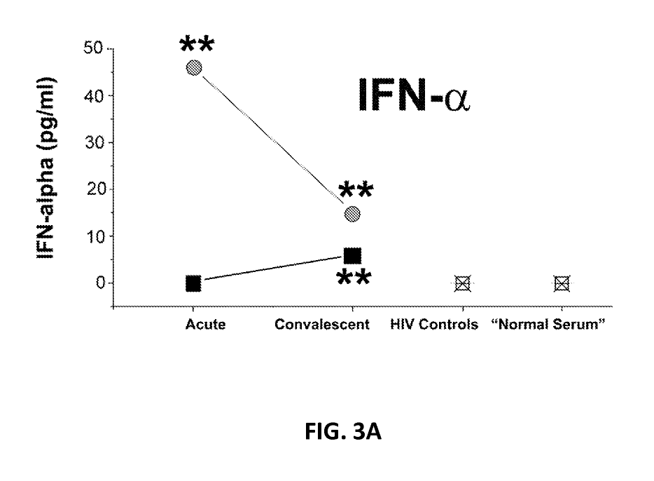

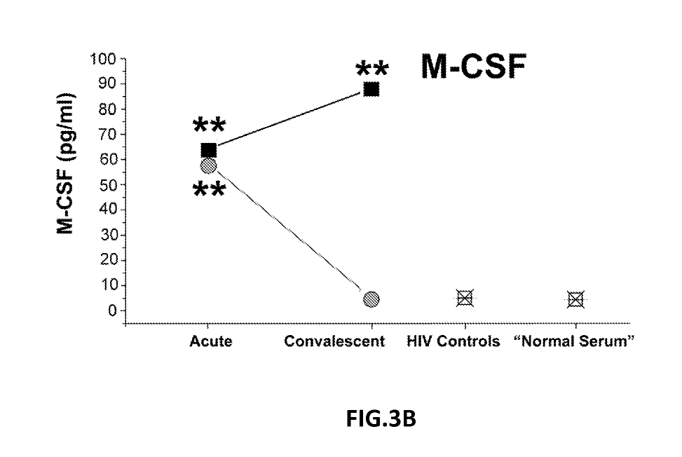

In the second category are analytes uniquely related to the severity of KLS. Referring now to FIGS. 2A and 2B. Analytes elevated in severe KLS (patient 1, black squares), but not typical KLS (patient 2, gray circles), compared to asymptomatic HIV⁺ control subjects. The control HIV subjects' mean (open squares) and range of analyte values are indicated in the third column. The level of analyte in a single HIV negative “normal serum” is shown as a square in the final column. For IL-1ra note the break in the scale, and that the level of IL-1ra in HIV negative “normal serum” is higher than in HIV⁺control subjects. IFN-γ and TNF-α were below the limit of detection for the HIV⁺ controls and “normal” serum; plotted as the lower limit of detection. Of these IFN-γ, CxCL10 [IP10], TNF-α, IL-10, IL-1ra and osteoprotegrin (OPG) were significantly elevated compared to asymptomatic HIV⁺ controls only in Patient 1 with KLS septic shock (FIG. 2A & 2B). Even though it carries a p value <0.05, CxCL10 was included in this group because the acute phase level in patient 2 was only 30% higher than the HIV⁺ control serum mean; IL-1ra was included because the acute phase level in patient 2 was lower than the IL-1ra level in the HIV negative “normal serum”. Conversely IFN-α was only elevated in Patient 2 with typical KLS (FIG. 3A); M-CSF was elevated in typical KLS, and persistently elevated in severe KLS (FIG. 3B). While conclusions cannot be drawn based on two patients, it is possible that the absence of an IFN-α response is a marker for severe KLS disease. With similar limitations, IL-1ra may be abnormally low in HIV⁺ individuals, without or with KLS, based on the higher level of IL-1ra in the HIV negative control serum.

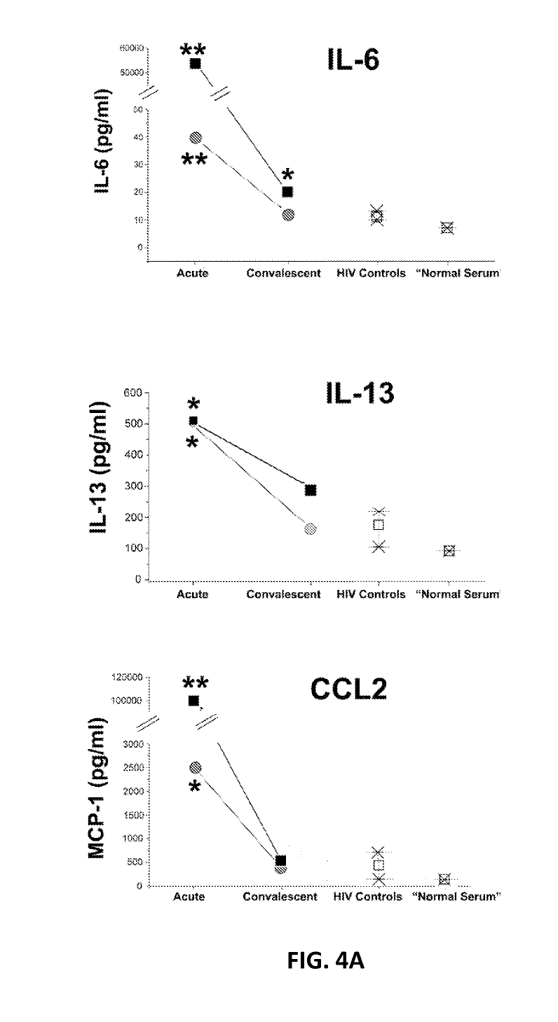

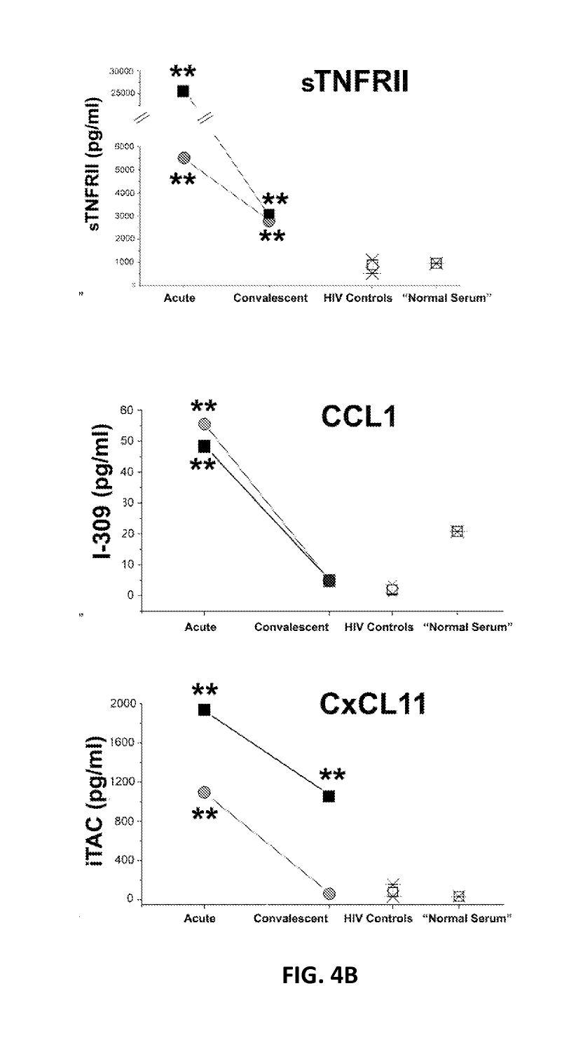

Referring now to FIGS. 4A and 4B. The third and most interesting category related to potential biomarkers, includes analytes elevated in KLS patients in the acute phase that, during the convalescent phase, decreased to or toward levels seen in asymptomatic HIV+ control subjects. The control HIV subjects' mean (open squares) and range of analyte values are indicated in the third column. The level of analyte in a single HIV negative “normal serum” is shown as a square in the final column. In this category are IL-6, sTNFRII, IL-13, CCL1 (I-309), CCL2 (MCP-1), and CxCL11 (I-TAC) (FIGS. 4A and 4B). As previously reported in pediatric KD, IL-6 and CCL2 were elevated in acute KLS; high levels of IL-6 have been associated with coronary artery aneurysm formation in pediatric KD. As previously reported in HIV KLS, sTNFRII was markedly elevated in both KLS patients in the acute phase. Particularly informative are significant elevations in the Th2 cytokine IL-13 and the Th2 chemokine CCL1 (I-309). Combined with the paucity of IFN-γ and CxCL10, these results argue that HIV KLS is a dysfunction Th2 inflammatory response to an unknown inciting agent. Finally, elevation of CxCL11 (I-TAC), an endothelial and vascular smooth muscle chemokine associated with recruitment of plasma cells, provides an immunobiological link in serum to the unique IgA⁺ plasma cell infiltration of vascular walls that is pathognomonic for KD/KLS.

Because HIV Kawasaki-like syndrome is a relatively rare disease the opportunity to study its pathophysiology is limited. Published investigations to date generally support the proposition that HIV KLS is the same disease as pediatric KD; occurring in adults likely made susceptible by immune dysfunction caused by advanced HIV disease (3). The clinical presentation of HIV KLS is similar to that of KD except that HIV patients do not typically have a sentinel lymph node and have lower ESR/CRP values than seen in pediatric KD patients. In HIV KLS patients the typical clinical presentation includes >5 days of fever, macular rash, conjunctivitis, painful swelling/erythema of the hands and feet, and changes of the oropharynx. Changes of the oropharynx fall on a spectrum of severity and can be complicated by co-morbidities such as thrush. Similarly, the rash can be subtle in individuals with dark skin pigments. Curiously, at least half of HIV individuals with KLS have mild abdominal pain with some degree of diarrhea preceding development of the protean manifestations of the syndrome. In this report, both KLS patients had an abdominal pain/diarrheal prodrome. Neither patient had a sentinel lymph node. Both patients had changes of the oropharynx, but that assessment was somewhat complicated in patient 2 because of co-existent thrush. A dark skin pigment made the rash in patient 2 a subtle physical finding. Because HIV KLS patients rarely, if ever, have a sentinel lymph node, there is risk that HIV KLS goes undiagnosed because affected individuals have less than the four physical findings required to make a formal HIV KLS diagnosis. Pending development of a diagnostic test for KD/KLS, HIV⁺ individuals with fevers >5 days, non-exudative conjunctivitis, changes in the peripheral extremities and no other source of fever should have HIV KLS considered as a possible etiology of their illness.

Patient 1 in this report was hospitalized with an ongoing febrile illness for >1 week before KLS was diagnosed and treated. This unintentional observation of the natural course of KLS in a patient with advanced HIV/AIDS offers new insights into KLS. Untreated KLS is potentially a fatal disease in HIV/AIDS. At the point that the IVIG infusion was started, Patient 1 required maximal fluid and pressor support to maintain his blood pressure. It is likely that he would have expired absent IVIG therapy as he was adrenally sufficient (random cortisol >20) and receiving hydrocortisone when his vascular tone collapsed with an echocardiogram that showed normal cardiac contractility. Because the patient was already on hydrocortisone when he developed severe hypotension, it seems unlikely that addition of methylprednisolone at the time of IVIG therapy was responsible for reversing the shock state. A previous investigation of HIV KLS noted recurrent fevers after an initial IVIG infusion, leading to retreatment with IVIG, plasmaphoresis, and thalidomide. In at least some patients, IVIG appears to reverse the protean manifestations of KLS but not the fever. The clinical course of Patient 1 highlights a role for aspirin in the treatment of HIV KLS. Fevers in Patient 1 did not break until aspirin therapy was initiated after the second IVIG infusion. It is possible that the second IVIG infusion would not have been necessary had he been able to take aspirin therapy with the first IVIG infusion. Based on the experience of patients 1 and 2 in this report, it appears that 325-650 mg aspirin po qid may be sufficient to achieve remission of the fevers, and that a drop down dose of 325 mg po bid is sufficient to sustain remission of fevers in the ˜1-2 week window of time immediately following IVIG treatment. The conventional dose of 80 mg/kg (3-4 full size aspirin tablets four times daily for an average size adult) does not appear to be necessary, and is viewed unfavorably by many adult medical practitioners. In children, low dose ASA (3-5 mg/kg daily) after IVIG appears to be sufficient therapy. Even though no coronary artery aneurysms have been documented in HIV KLS patients, possibly because most diagnosed cases get treated with IVIG and few have angiography, that outcome and KLS septic shock (this report) are likely preventable with IVIG treatment, and IVIG probably should be administered beyond the recommended 10 day febrile window used in pediatrics. Early diagnosis and treatment of KLS with IVIG and aspirin are important for maximizing patient well-being and limiting medical expenditures on prolonged hospitalizations for fevers of unknown origin. Based on lack of a convenient modality for assessing coronary artery pathology in adults (transthoracic echocardiography is not adequate to visualize adult coronary arteries), after the initial 2 weeks of ASA therapy it seems reasonable to continue HIV KLS patients on low dose ASA (81-165 mg daily) until a follow up cardiac stress test 6-12 months out from the acute illness rules out ischemic complications. Limited pediatric KD data suggest that negative myocardial perfusion cardiac stress testing has a reasonable negative predictive value for future cardiac events.

The hemodynamic collapse in patient 1 was due to loss of vascular tone as described with pediatric Kawasaki Disease Septic Shock (KDSS); this case was likely the adult HIV KLS equivalent of KDSS. Multiplex ELISA definition of the cytokine milieu in Patient 1 supports loss of vascular tone and increased capillary permeability (CXR had increased interstitial markings) due to a cytokine storm that included high levels of TNFα and IL-6, coupled with an ineffective elevation of counter inflammatory cytokines IL-10 and IL-1ra. Relative level of IL-10 (14 pg/ml) was low in the septic state, and the high level of IL-1ra may not have been of much benefit as surprisingly the serum level of IL-1βin both KLS patients was <0.4 pg/ml (see Table 3). TNFα appears to be a central cytokine in KLS as sTNFRII was markedly elevated in both KLS patients, even when TNFα was not directly measurable in the serum (Patient 2). Elevated sTNFRII has previously been reported in a HIV⁺ patient with a Kawasaki Disease like illness. The two additional HIV KLS patients in this report with elevated sTNFRII in the acute phase reinforce the hypothesis that TNFα is a critical cytokine in the pathophysiology of KLS as soluble TNF receptors levels in serum are regulated by TNFα. In one pediatric KD report, patients with the highest serum TNF-α serum levels had an increased risk of coronary aneurysm formation, and TNFRI has previously be reported to be elevated in pediatric patients with KD.

HIV KLS appears to be pauci-interferon vasculitis, but whether the same is true in pediatric KD is less clear. Pediatric KD studies found low to undetectable serum levels of type 1 and type II interferons, and a decreased frequency of intracellular IFN-γ⁺ circulating T lymphocytes in acute pediatric KD. In this report, Patient 2 with typical KLS had elevated levels of IFN-α (46 pg/ml) while IFN-γ was below the limit of detection (<10 pg/ml). In patient 1 during KLS septic shock, IFN-α was undetectable and IFN-γ was elevated (27 pg/ml). The serum IFN-γ level in normal serum is undetectable and during acute viral illnesses (Influenza, RSV, Adenovirus, Dengue fever) has been reported to run in the 8-30 pg/ml range; serum IFN-α levels in children with influenza has a reported mean value of 602+/−95 pg/ml. A more informative global readout of interferon biology is likely CxCL10, a chemokine up regulated by type 1 and 2 interferons. Serum levels of CxCL10 have been reported to be up regulated by two groups studying acute pediatric KD patients (see Table 2); in this report CxCL10 was not remarkably elevated in either KLS patient in the acute phase of KLS.

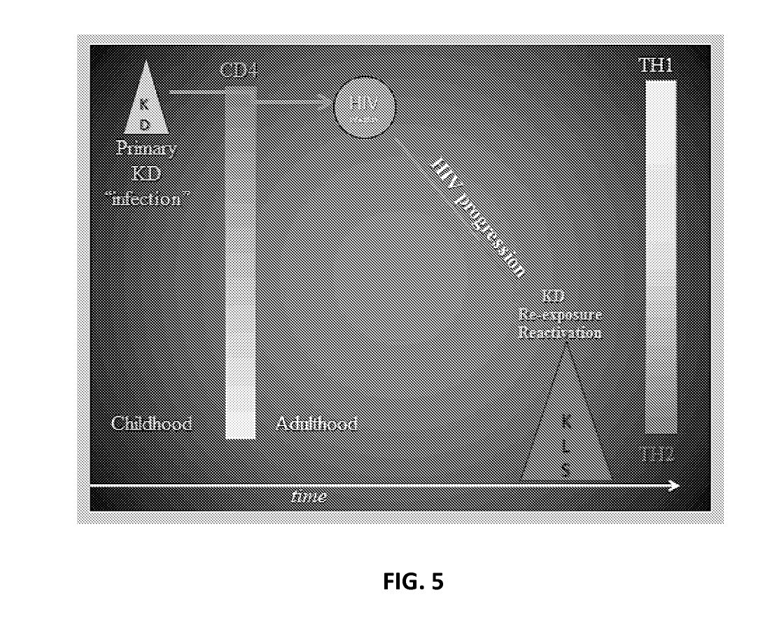

As reported herein elevated levels of the Th2 cytokine IL-13 and the Th2 chemokine CCL1 (I-309) isolated from the serum of both adult HIV KLS patients in the acute phase that, in the convalescent phase, returned to levels found in asymptomatic HIV⁺ controls. In pediatric KD, others have documented excess serum IL-4 (median acute KD 17 pg/ml versus 7.6 pg/ml healthy controls) and minimally elevated levels of IFN-γ (median in acute KD 0.6 units/ml versus healthy control 0.4 units/ml). Patients with advanced HIV appear to drift away from Th1 responses toward Th2 responses, with progressive loss of CD4 T cell frequency/production of IL-2 & IFN-γ with relative preservation of CD4 T cell IL-4 frequency/production, though not all studies support this Th1 to Th2 shift with HIV progression. Functional evidence in support of a Th2 shift with HIV progression includes development of a pathogen-independent eosinophilia (Th2 cytokine IL-5) in some patients with declining CD4 T cell counts; idiopathic eosinophilia was a pre-existing condition in Patient 2 of this report. These data support a KLS model in which a ubiquitous infectious agent acquired either through re-exposure or reactivation triggers a pathologic Th2 response in HIV⁺ individuals due to HIV-related diminution in Th1IFN-γ/IL-2 responses caused by progression of HIV disease (FIG. 5). Recurrent episodes of KLS 5 months to 2 years after resolution of a primary KLS episode have been documented in HIV⁺ individuals, suggesting that reactivation of a latent “infection” is more likely than re-exposure. Lack of a sentinel lymph node in HIV KLS patients may also be a manifestation of a reactivated rather than primary infection.

Referring now to FIG. 5. Model of HIV KLS. A nondescript KD “infection” occurs in early childhood in the setting of a healthy Th1 cellular immune response that controls the infection without development of clinical Kawasaki Disease. In adulthood, acquisition of HIV leads to a steady decline in CD4 T cell numbers and a shift toward Th2 immune responses. After significant degradation of memory T cell immunity, the KD agent is able to reactivate. Unlike the original protective Th1 response, the secondary response to the KD agent is a less effective Th2 response that results in the physical manifestations of KLS.

In general, the cytokine milieu in the HIV KLS patient with a typical presentation (patient 2) was consistent with that previously reported in pediatric KD including elevations in CCL2 (MCP-1), IL-6, and M-CSF, TNFα related biology (TNFRII) (see Table 3). The differences between typical KLS and pediatric KD were the less-than-significant elevations in osteoprotegrin and IL-10, and lack of an IL-1β and IL-17 response. It is problematic to emphasize comparisons based on non-standardized testing; for example the reported IL-1β levels are based on a radioimmunoassay of 6 KD patients with 5 healthy controls in 1988. Elevations in CCL2, IL-6, and TNF-a biology (TNF-a, sTNFR1) have been reported in two or more KD studies. Elevations of CCL2, IL-6, and TNFRII in the two KLS patients in this report, along with the previous demonstration of IgA infiltration of vessel walls on conjunctival bx during acute HIV KLS support a similar pathophysiology and likely inciting agent in KLS and KD. The IgA antibody response in pediatric KD is oligoclonal, and IgA monoclonal antibodies reverse-engineered from KD patients recognize RNA-containing cytoplasmic inclusions in KD patient tissues. It has previously been postulated that the ontogeny of KD IgA plasma cells includes original expansion in the pulmonary or gastrointestinal mucosa. Without being bound by theory of hypothesis, it is noted that these results fit nicely with the theory that HIV KLS patients commonly having an abdominal pain/diarrhea prodrome before developing the protean manifestations of KLS, and the RNA-containing cytoplasmic inclusions with the possibility of a latent “infection”.

The cytokine milieu in KLS patients during the acute vasculitis strongly corroborates the known findings of inflammation within vascular walls including IgA⁺ plasma cells. CCL2 (MCP-1), a chemokine made by vascular smooth muscle cells and endothelium, is dramatically elevated in KLS. CCL2 is a well-established biomarker for atherosclerosis, an inflammatory condition within arterial walls. CCL1 (I-309), an endothelial and smooth muscle chemokine, recruits Th2 T lymphocytes via chemokine receptor 8 (CCR8), supports KLS being a Th2-type vasculitis. The Th2 conclusion is further supported by readily measurable levels of the Th2 cytokine IL-13 in both KLS patients in this report, in the absence or near absence of IFN-γ (a Th2 cytokine) and CxCL10 (regulated by IFN-γ). More interesting, CxCL11, a cytokine that recruits plasma cells via chemokine receptor CxCR3, was markedly elevated in both KLS patients, while levels in asymptomatic HIV control subjects and the “normal serum” approximated 0. In human peripheral blood, majorities of IgA⁺ memory B cells and IgA⁺ antibody secreting cells (ASC, i.e. plasma cells) are CxCR3 positive. In humans elevated CxCL11 is a biomarker associated with aortic aneurysm formation, and its receptor CxCR3 is required for aneurysm formation in a mouse model. CxCR3 inhibitors are being developed and have entered early human clinical trials. Based on limited data, it appears that the KD/KLS pathognomonic finding of IgA plasma cell infiltration of arterial walls is reflected in the blood in the form of the plasma cell recruiting chemokine CxCL11. The KLS serum cytokine data in this report support a pathologic role for Th2 type inflammation and CxCL11 in recruitment of plasma cells during the acute phase of KLS. The intimate relationship between the serum and inflamed vascular wall raises the possibility of a diagnostic test for KLS and KD based on serum analytes; perhaps a combination of elevated CxCL11, CCL1, CCL2, IL-6, IL-13 and TNFRII, or possibly as simple as elevated CCL1 (Th2 inflammation) and CxCL11 (plasma cell recruitment) in the right clinical setting.

TABLE 1
Diagnostic Testing

| Diagnostic Test | Patient 1 | Patient 2 |
|---|---|---|
| Admission WBC | 5.8 | 3.5 |
| CD4 count | 3 | 19 |
| HIV vl | 180,000 | 139,000 |
| HCV studies | 1a | neg |
| HBV sAg | neg | neg |
| Oral HSV culture | neg | ND |
| CXR | NAD | NAD |
| Urinalysis | 2 WBC | 0 WBC |
| CSF | ND | 1 WBC |
| ESR | 5 | 87 |
| ANA | ND | <1:40 |
| LDH | 1370 | ND |
| Ig A level | 531 | 267 |
| ASO titer (ULN <199) | 200 | 200 |
| Strep screen | neg | ND |
| RPR | neg | neg |
| Monospot | neg | neg |
| EBV IgM | neg | neg |
| CMV IgM | neg | neg |
| Toxo IgM | neg | ND |
| Erhlichea pcr | neg | ND |
| Parvovirus B19 pcr | neg | ND |
| Parvovirus B19 IgM | ND | neg |
| HHV6 pcr | neg | ND |
| Urine Histoplasma ag | neg | neg |
| Serum cryptococcal ag | ND | neg |
| Blood cultures | neg | neg |
| Stool culture/O&P | neg | neg |
| Chlamydia/GC LCR | ND | neg |
| Nasopharynx viral cult. | neg | neg |
| BAL stains | neg | ND |
| BAL culture | few | ND |
| | Candida | |
| BM bx stains & cultures | neg | ND |
| Skin bx | perivascular | perivascular |
| | lymphoctyes | lymphocytes, |
| | | few eosinophils |

NAD = no active disease; ND = not determined.

TABLE 2
Analyte Panel

| Analyte | Cell type origin | Ref. | KD serum | KD ref. |
|---|---|---|---|---|
| IL-1beta | Multiple | (4) | Elevated | (5) |
| IL-1ra | Multiple excepting lymphocytes | (6) | No data | NA |
| IL-6 | Multiple including T cells | (7, 8) | Elevated | (9-11) |
| IL-10 | Hematopoetic cells including T cells | (12) | Elevated | (13, 14) |
| IL-13 | T cells (Th2), eosinophils, mast cells | (15-17) | No data | NA |
| IL-17 | T cells (Th17 & some Th1, rare | (18) | Elevated | (19) |
| | CD8) | | | |
| IFN-α | Multiple | | Conflicting | (20, 21) |
| IFN-γ | T cells (Th1) | (22) | Not elevated | (23, 24) |
| | | | In majority | |
| TNFα | Multiple including T cells | (25) | Elevated | (10, 26, 27) |
| sTNFRII | Multiple, regulated by TNFα | (28) | Elevated KD | (3, 26) |
| | | | & KLS | |
| M-CSF | Mesenchymal cell types | (29) | Elevated | (30, 31) |
| | (endothelium, fibroblasts) | | | |
| Osteoprotegrin | Osteoblasts, endothelial and smooth | (32) | Elevated | (33) |
| | muscle cells | | | |
| CCL1 (I-309) | Monocytes, T cells, endothelial | (34-37) | No data | NA |
| | cells; recruits Th2 T cells, | | | |
| | monocytes, endothelial and smooth | | | |
| | muscle cells | | | |
| CCL2 (MCP-1) | Monocytes & stromal cells | (38) | Elevated | (39-41) |
| | (epithelium, endothelium, | | | |
| | fibroblasts, smooth muscle cells) | | | |
| CCL5 (Rantes) | Multiple | (42) | Elevated | (43) |
| CxCL10 (IP-10) | Multiple, regulated by interferons | (44) | Elevated | (41) |
| CxCL11 (I-TAC) | Endothelial, epithelial cells and | (45, 46) | No data | NA |
| | leukocytes; recruitment of T cells | | | |
| | and non-homeostatic recruitment of | | | |
| | plasma cells | | | |

NA = not applicable

TABLE 3
KLS/KD Acute Phase Analytes

| | This report | | | | | | |
|---|---|---|---|---|---|---|---|
| | HIV controls | | | | | | |
| Analyte | HIV KLS | | | KD | Other | Other | |
| (pg/ml) | Mean P1, P2 | | Control | (KLS) | disease 1 | disease 2 | Reference |
| IL-1beta | <0.4 | <0.4, <0.4 | 310 | 1110 | — | — | (5) |
| IL-1ra | 59 | 25050, 882 | 410 | no data | 7400 | — | (47) |
| | | | | | (urosepsis) | | |
| IL-6 | 12 | 54000, 40 | <5 | 123 | 26 | — | (11) |
| | | | | | (enterovirus) | | |
| | | | 1.5 | 164 | 6 | — | (10) |
| | | | | | (febrile illness) | | |
| IL-10 | 1.9 | 14, 1.4 | 16 | 125 | 27 | — | (48) |
| | | | | | (febrile illness) | | |
| | | | 4 | 122 | 34 | — | (13) |
| | | | | | (febrile illness) | | |
| IL-13 | 176 | 510, 502 | 35 | no data | 57 | — | (49) |
| | | | | | (RSV) | | |
| IL-17 | 171 | 171, 191 | 2 | 25 | — | — | (50) |
| IFN-α^{‡} | <0.8 | <0.8, 46 | — | — | — | — | — |
| IFN-γ^{§} | <10 | 27, <10 | — | — | — | — | — |
| TNFα | <5 | 60, <5 | 10 | 24 | — | — | (27) |
| | | | <3.4 | 8 | <3.4 | 12 | (51) |
| | | | | | (measles) | (anaphylaxis) | |
| sTNFRI | — | — | — | 2750000 | 1550000 | — | (52) |
| | | | | | (encephalitis) | | |
| sTNFRII | 860 | 25700, 5540 | — | (>5000) | — | — | (3) |
| M-CSF* | <16 | 62, 58 | — | — | — | — | — |
| Osteoprotegrin | 2 | 28, 9 | 40 | 101 | 68 | 80 | (33) |
| | | | | | (JSLE) | (febrile illness) | |
| CCL1 (1-309) | 1.9 | 48, 56 | — | — | — | — | — |
| CCL2 (MCP-1) | 336 | 100200, 2510 | — | 443 | 83 | 328 | (41) |
| | | | | | (HSP) | (febrile illness) | |
| | | | 223 | 829 | — | — | (53) |
| | | | 290 | 1320 | — | — | (40) |
| CCL5 (RANTES)† | 53833 | 15600, 156600 | — | — | — | — | — |
| CxCL10 (IP-10) | 52 | 126, 73 | — | 538 | 59 | 417 | (41) |
| | | | | | (HSP) | (febrile illness) | |
| | | | 128 | 2469 | — | — | (53) |
| CxCL11 (I-TAC) | 48 | 1935, 1100 | 68 | no data | 254 | — | (54) |
| | | | | | (HCV | | |
| | | | | | cryoglobulinemia) | | |

P1 = patient 1;
P2 = patient 2;
^{‡}conflicting data based on bioassays;
^{§}published data in units/ml without conversion factor;
*published data in units/ml without conversion factor;
^{†}published data in arbitrary units mRNA in peripheral blood mononuclear cells (PBMC.

TABLE 4
A summary of protein analysis results; the levels of 16 different
proteins were measured in 9 samples of human serum.

| Test | Sample | hIL13 | hMCP1 | hIL6 | hIL17 |
|---|---|---|---|---|---|
| ID | ID | pg/ml | pg/ml | pg/ml | pg/ml |
| 1 | #1 | 510.4 | 100200.0 | 54000.0 | 171.2 |
| 2 | #2 | 286.0 | 566.8 | 23.0 | 458.2 |
| 3 | #3 | 502.4 | 2510.0 | 40.0 | 190.8 |
| 4 | #4 | 163.6 | 358.0 | 11.8 | 107.4 |
| 5 | #5 | 204.0 | 361.0 | 11.0 | 132.0 |
| 6 | #6 | 218.6 | 529.8 | 11.0 | 250.8 |
| 7 | #7 | 104.2 | 115.6 | 12.6 | 130.2 |
| 8 | #8 | 93.6 | 161.2 | 7.4 | 100.0 |
| Test | Sample | hTNFRII | hIP10 | hRANTES | hITAC |
| ID | ID | pg/ml | pg/ml | pg/ml | pg/ml |
| 1 | #1 | 25700.0 | 125.8 | 15700.0 | 1935.0 |
| 2 | #2 | 3120.0 | 70.8 | 34000.0 | 1050.5 |
| 3 | #3 | 5540.0 | 72.6 | 156600.0 | 1099.5 |
| 4 | #4 | 2765.0 | 56.4 | 30300.0 | 59.2 |
| 5 | #5 | 955.0 | 51.2 | 86200.0 | 17.8 |
| 6 | #6 | 690.0 | 51.0 | 29700.0 | 21.8 |
| 7 | #7 | 934.6 | 55.0 | 45600.0 | 104.6 |
| 8 | #8 | 975.1 | 51.2 | 39700.0 | 10.0 |
| Test | Sample | hOPG | hM-CSF | hTNFa | hIL10 |
| ID | ID | pg/ml | pg/ml | pg/ml | pg/ml |
| 1 | #1 | 27.8 | 62.4 | 60.4 | 14.4 |
| 2 | #2 | 8.4 | 88.0 | <4.7 | 2.4 |
| 3 | #3 | 9.4 | 58.4 | <4.7 | 1.4 |
| 4 | #4 | 1.2 | <15.6 | <4.7 | 0.4 |
| 5 | #5 | 0.8 | <15.6 | <4.7 | 0.8 |
| 6 | #6 | 0.4 | <15.6 | <4.7 | 0.6 |
| 7 | #7 | 4.0 | <15.6 | <4.7 | 4.2 |
| 8 | #8 | 0.8 | <15.6 | <4.7 | 1.2 |
| Test | Sample | hIL1ra | hI309 | hIFNa | hIL1b |
| ID | ID | pg/ml | pg/ml | pg/ml | pg/ml |
| 1 | #1 | 25050.0 | 48.4 | <0.8 | <0.4 |
| 2 | #2 | 361.6 | 4.8 | 5.8 | <0.4 |
| 3 | #3 | 882.8 | 55.6 | 46.2 | <0.4 |
| 4 | #4 | 38.8 | 4.8 | 14.8 | <0.4 |
| 5 | #5 | <15.6 | <1.6 | <0.8 | <0.4 |
| 6 | #6 | 33.6 | <1.6 | <0.8 | <0.4 |
| 7 | #7 | 134.4 | 3.6 | <0.8 | <0.4 |
| 8 | #8 | 2048.8 | 20.8 | <0.8 | <0.4 |

### REFERENCES

1. Johnson, R. M., J. R. Little, and G. A. Storch. 2001. Kawasaki-like syndromes associated with human immunodeficiency virus infection. Clin Infect Dis 32: 1628-1634.
2. Rowley, A. H., C. A. Eckerley, H. M. Jack, S. T. Shulman, and S. C. Baker. 1997. IgA plasma cells in vascular tissue of patients with Kawasaki syndrome. J Immunol 159: 5946-5955.
3. Blanchard, J. N., H. C. Powell, W. R. Freeman, S. Letendre, D. Blanchard, C. Shimizu, and J. C. Burns. 2003. Recurrent Kawasaki disease-like syndrome in a patient with acquired immunodeficiency syndrome. Clin Infect Dis 36: 105-111.
4. Dinarello, C. A. 1988. Biology of interleukin 1. FASEB J 2: 108-115.
5. Maury, C. P., E. Salo, and P. Pelkonen. 1988. Circulating interleukin-1 beta in patients with Kawasaki disease. N Engl J Med 319: 1670-1671.
6. Arend, W. P., M. Malyak, C. J. Guthridge, and C. Gabay. 1998. Interleukin-1 receptor antagonist: role in biology. Annu Rev Immunol 16: 27-55.
7. Gabay, C. 2006. Interleukin-6 and chronic inflammation. Arthritis Res Ther 8 Suppl 2: S3.
8. Gattorno, M., P. Facchetti, F. Ghiotto, S. Vignola, A. Buoncompagni, I. Prigione, P. Picco, and V. Pistoia. 1997. Synovial fluid T cell clones from oligoarticular juvenile arthritis patients display a prevalent Th1/Th0-type pattern of cytokine secretion irrespective of immunophenotype. Clin Exp Immunol 109: 4-11.
9. Ueno, Y., N. Takano, H. Kanegane, T. Yokoi, A. Yachie, T. Miyawaki, and N. Taniguchi. 1989. The acute phase nature of interleukin 6: studies in Kawasaki disease and other febrile illnesses. Clin Exp Immunol 76: 337-342.
10. Gupta, M., G. J. Noel, M. Schaefer, D. Friedman, J. Bussel, and R. Johann-Liang. 2001. Cytokine modulation with immune gamma-globulin in peripheral blood of normal children and its implications in Kawasaki disease treatment. J Clin Immunol 21: 193-199.
11. Kim, D. S. 1992. Serum interleukin-6 in Kawasaki disease. Yonsei Med J 33: 183-188.
12. Moore, K. W., A. O'Garra, R. de Waal Malefyt, P. Vieira, and T. R. Mosmann. 1993. Interleukin-10. Annu Rev Immunol 11: 165-190.
13. Kim, D. S., H. K. Lee, G. W. Noh, S. I. Lee, and K. Y. Lee. 1996. Increased serum interleukin-10 level in Kawasaki disease. Yonsei Med J 37: 125-130.
14. Okada, Y., M. Shinohara, T. Kobayashi, Y. Inoue, T. Tomomasa, and A. Morikawa. 2003. Effect of corticosteroids in addition to intravenous gamma globulin therapy on serum cytokine levels in the acute phase of Kawasaki disease in children. J Pediatr 143: 363-367.
15. Minty, A., P. Chalon, J. M. Derocq, X. Dumont, J. C. Guillemot, M. Kaghad, C. Labit, P. Leplatois, P. Liauzun, B. Miloux, and et al. 1993. Interleukin-13 is a new human lymphokine regulating inflammatory and immune responses. Nature 362: 248-250.
16. Jaffe, J. S., D. G. Raible, T. J. Post, Y. Wang, M. C. Glaum, J. H. Butterfield, and E. S. Schulman 1996. Human lung mast cell activation leads to IL-13 mRNA expression and protein release. Am J Respir Cell Mol Biol 15: 473-481.
17. Schmid-Grendelmeier, P., F. Altznauer, B. Fischer, C. Bizer, A. Straumann, G. Menz, K. Blaser, B. Wuthrich, and H. U. Simon. 2002. Eosinophils express functional IL-13 in eosinophilic inflammatory diseases. J Immunol 169: 1021-1027.
18. Peck, A., and E. D. Mellins. Plasticity of T-cell phenotype and function: the T helper type 17 example. Immunology 129: 147-153.
19. Jia, S., C. Li, G. Wang, J. Yang, and Y. Zu. The T helper type 17/regulatory T cell imbalance in patients with acute Kawasaki disease. Clin Exp Immunol 162: 131-137.
20. Rowley, A. H., S. T. Shulman, O. T. Preble, B. J. Poiesz, G. D. Ehrlich, and J. R. Sullivan. 1988. Serum interferon concentrations and retroviral serology in Kawasaki syndrome. Pediatr Infect Dis J 7: 663-666.
21. Ogle, J. W., J. L. Waner, L. S. Joffe, R. L. Brogden, J. Wiggins, and M. P. Glode. 1991. Absence of interferon in sera of patients with Kawasaki syndrome. Pediatr Infect Dis J 10: 25-29.
22. Mosmann, T. R., and R. L. Coffman. 1989. TH1 and TH2 cells: different patterns of lymphokine secretion lead to different functional properties. Annu Rev Immunol 7: 145-173.
23. Matsubara, T., S. Furukawa, and K. Yabuta. 1990. Serum levels of tumor necrosis factor, interleukin 2 receptor, and interferon-gamma in Kawasaki disease involved coronary-artery lesions. Clin Immunol Immunopathol 56: 29-36.
24. Matsubara, T., K. Katayama, T. Matsuoka, M. Fujiwara, M. Koga, and S. Furukawa. 1999. Decreased interferon-gamma (IFN-gamma)-producing T cells in patients with acute Kawasaki disease. Clin Exp Immunol 116: 554-557.
25. Darrah, P. A., D. T. Patel, P. M. De Luca, R. W. Lindsay, D. F. Davey, B. J. Flynn, S. T. Hoff, P. Andersen, S. G. Reed, S. L. Morris, M. Roederer, and R. A. Seder. 2007. Multifunctional TH1 cells define a correlate of vaccine-mediated protection against Leishmania major. Nat Med 13: 843-850.
26. Furukawa, S., T. Matsubara, Y. Umezawa, K. Okumura, and K. Yabuta. 1994. Serum levels of p60 soluble tumor necrosis factor receptor during acute Kawasaki disease. J Pediatr 124: 721-725.
27. Ahn, S. Y., G. C. Jang, J. S. Shin, K. M. Shin, and D. S. Kim. 2003. Tumor necrosis factor-alpha levels and promoter polymorphism in patients with Kawasaki disease in Korea. Yonsei Med J 44: 1021-1026.
28. Diez-Ruiz, A., G. P. Tilz, R. Zangerle, G. Baier-Bitterlich, H. Wachter, and D. Fuchs. 1995. Soluble receptors for tumour necrosis factor in clinical laboratory diagnosis. Eur J Haematol 54: 1-8.
29. Sweet, M. J., and D. A. Hume. 2003. CSF-1 as a regulator of macrophage activation and immune responses. Arch Immunol Ther Exp (Warsz) 51: 169-177.
30. Igarashi, H., K. Hatake, H. Tomizuka, M. Yamada, Y. Gunji, and M. Y. Momoi. 1999. High serum levels of M-CSF and G-CSF in Kawasaki disease. Br J Haematol 105: 613-615.
31. Oana, S., M. Terai, and Y. Kohno. 1999. Serum M-CSF levels in Kawasaki disease. Br J Haematol 107: 462-463.
32. Hofbauer, L. C., C. Shui, B. L. Riggs, C. R. Dunstan, T. C. Spelsberg, T. O'Brien, and S. Khosla. 2001. Effects of immunosuppressants on receptor activator of NF-kappaB ligand and osteoprotegerin production by human osteoblastic and coronary artery smooth muscle cells. Biochem Biophys Res Commun 280: 334-339.
33. Simonini, G., L. Masi, T. Giani, E. Piscitelli, R. Cimaz, S. Vierucci, M. L. Brandi, and F. Falcini. 2005. Osteoprotegerin serum levels in Kawasaki disease: an additional potential marker in predicting children with coronary artery involvement. J Rheumatol 32: 2233-2238.
34. Miller, M. D., S. Hata, R. De Waal Malefyt, and M. S. Krangel. 1989. A novel polypeptide secreted by activated human T lymphocytes. J Immunol 143: 2907-2916.
35. Miller, M. D., S. D. Wilson, M. E. Dorf, H. N. Seuanez, S. J. O'Brien, and M. S. Krangel. 1990. Sequence and chromosomal location of the 1-309 gene. Relationship to genes encoding a family of inflammatory cytokines. J Immunol 145: 2737-2744.
36. Zingoni, A., H. Soto, J. A. Hedrick, A. Stoppacciaro, C. T. Storlazzi, F. Sinigaglia, D. D'Ambrosio, A. O'Garra, D. Robinson, M. Rocchi, A. Santoni, A. Zlotnik, and M. Napolitano. 1998. The chemokine receptor CCR8 is preferentially expressed in Th2 but not Th1 cells. J Immunol 161: 547-551.
37. Hague, N. S., J. T. Fallon, J. J. Pan, M. B. Taubman, and P. C. Harpel. 2004. Chemokine receptor-8 (CCR8) mediates human vascular smooth muscle cell chemotaxis and metalloproteinase-2 secretion. Blood 103: 1296-1304.
38. Deshmane, S. L., S. Kremlev, S. Amini, and B. E. Sawaya. 2009. Monocyte chemoattractant protein-1 (MCP-1): an overview. J Interferon Cytokine Res 29: 313-326.
39. Terai, M., T. Jibiki, A. Harada, Y. Terashima, K. Yasukawa, S. Tateno, H. Hamada, S. Oana, H. Niimi, and K. Matsushima. 1999. Dramatic decrease of circulating levels of monocyte chemoattractant protein-1 in Kawasaki disease after gamma globulin treatment. J Leukoc Biol 65: 566-572.
40. Asano, T., and S. Ogawa. 2000. Expression of monocyte chemoattractant protein-1 in Kawasaki disease: the anti-inflammatory effect of gamma globulin therapy. Scand J Immunol 51: 98-103.
41. Chung, H. S., H. Y. Kim, H. S. Kim, H. J. Lee, J. H. Yuh, E. S. Lee, K. H. Choi, and Y. H. Lee. 2004. Production of chemokines in Kawasaki disease, Henoch-Schonlein purpura and acute febrile illness. J Korean Med Sci 19: 800-804.
42. Olson, T. S., and K. Ley. 2002. Chemokines and chemokine receptors in leukocyte trafficking. Am J Physiol Regul Integr Comp Physiol 283: R7-28.
43. Wong, M., E. D. Silverman, and E. N. Fish. 1997. Evidence for RANTES, monocyte chemotactic protein-1, and macrophage inflammatory protein-1 beta expression in Kawasaki disease. J Rheumatol 24: 1179-1185.
44. Liu, M., S. Guo, J. M. Hibbert, V. Jain, N. Singh, N. O. Wilson, and J. K. Stiles. CXCL10/IP-10 in infectious diseases pathogenesis and potential therapeutic implications. Cytokine Growth Factor Rev 22: 121-130.
45. Cole, K. E., C. A. Strick, T. J. Paradis, K. T. Ogborne, M. Loetscher, R. P. Gladue, W. Lin, J. G. Boyd, B. Moser, D. E. Wood, B. G. Sahagan, and K. Neote. 1998. Interferon-inducible T cell alpha chemoattractant (I-TAC): a novel non-ELR CXC chemokine with potent activity on activated T cells through selective high affinity binding to CXCR3. J Exp Med 187: 2009-2021.
46. Johansson, C., I. Ahlstedt, S. Furubacka, E. Johnsson, W. W. Agace, and M. Quiding-Jarbrink. 2005. Differential expression of chemokine receptors on human IgA+ and IgG+ B cells. Clin Exp Immunol 141: 279-287.
47. Olszyna, D. P., J. M. Prins, B. Buis, S. J. van Deventer, P. Speelman, and T. van der Poll. 1998. Levels of inhibitors of tumor necrosis factor alpha and interleukin lbeta in urine and sera of patients with urosepsis. Infect Immun 66: 3527-3534.
48. Noh, G. W., W. G. Lee, W. Lee, and K. Lee. 1998. Effects of intravenous immunoglobulin on plasma interleukin-10 levels in Kawasaki disease. Immunol Lett 62: 19-24.
49. Chung, H. L., H. J. Park, S. Y. Kim, and S. G. Kim. 2007. Age-related difference in immune responses to respiratory syncytial virus infection in young children. Pediatr Allergy Immunol 18: 94-99.
50. Sohn, M. H., S. Y. Noh, W. Chang, K. M. Shin, and D. S. Kim. 2003. Circulating interleukin 17 is increased in the acute stage of Kawasaki disease. Scand J Rheumatol 32: 364-366.
51. Furukawa, S., T. Matsubara, K. Yone, Y. Hirano, K. Okumura, and K. Yabuta. 1992. Kawasaki disease differs from anaphylactoid purpura and measles with regard to tumour necrosis factor-alpha and interleukin 6 in serum. Eur J Pediatr 151: 44-47.
52. Korematsu, S., S. Uchiyama, H. Miyahara, T. Nagakura, N. Okazaki, T. Kawano, M. Kojo, and T. Izumi. 2007. The characterization of cerebrospinal fluid and serum cytokines in patients with Kawasaki disease. Pediatr Infect Dis J 26: 750-753.
53. Shikishima, Y., T. Saeki, and N. Matsuura. 2003. Chemokines in Kawasaki disease: measurement of CCL2, CCL22 and CXCL10. Asian Pac J Allergy Immunol 21: 139-143.
54. Antonelli, A., C. Ferri, S. M. Ferrari, I. Ruffilli, M. Colaci, S. Frascerra, M. Miccoli, F. Franzoni, F. Galetta, and P. Fallahi. High serum levels of CXCL11 in mixed cryoglobulinemia are associated with increased circulating levels of interferon-gamma J Rheumatol 38: 1947-1952.

While the novel technology has been illustrated and described in detail in the figures, tables, and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

1-18. (canceled)

19. A method for treating a patient based on the unique pathophysiology of KLS/KD, comprising the steps of:
quantifying at least one human protein in a serum sample from a patient, the protein is selected from the group consisting of: IL-6, IL-13, sTNFRII, CCL1 (I-309), CCL2 (MCP-1), and CxCL11 (I-TAC), wherein the patient exhibits at least one symptom of Kawasaki disease or KLS;
measuring the level of “plasma cell recruiting” chemokine CxCL11 and/or “Th2” biomarkers IL-13 & CCL1 (singly or in combination) and/or “vascular inflammation marker” CCL2 in the serum sample; and
tagging the sample as being from a patient positive or preliminarily positive for KD or HIV KLS if the level of “plasma cell recruiting” chemokine CxCL11 and/or “Th2” biomarkers IL-13 & CCL1 (singly or in combination) and/or “vascular inflammation marker” CCL2 isolated from the sample is 2 or more Standard Deviations higher than are the levels of these proteins in the serum of a similar patient, wherein the similar patient is asymptomatic for KLS and KD.

20. The method according to claim 19, further including the step of:
validating that the patient is positive for KD or HIV KLS by confirming that the levels of sTNRII and/or IL-6 quantified in the patients' sample is 2 or more Standard Deviations higher than the levels of these proteins isolated from the serum of a similar patient, wherein the similar patient is asymptomatic for KLS and KD.

21. The method according to claim 19, wherein the quantifying and/or measuring steps includes the use of a Multiplex ELISA.

22. The method according to claim 20, wherein the quantifying and/or measuring steps includes the use of a Multiplex ELISA.

23. The method according to claim 19, wherein the quantifying and/or measuring steps include the use of a strip testing technology.

24. The method according to claim 20, wherein the quantifying and/or measuring steps include the use of a strip testing technology.

25. The method according to claim 19, wherein the quantifying and/or measuring steps include the use of a mass spectroscopy identification technique.

26. A treatment method, comprising the steps of:
administering a therapeutically effective dose of at least one compound that interferes with the interactions of at least one receptor ligand pair selected from the group consisting of: CxCL11-CCR3, CCL1-CCR8, and CCL2-CCR2 wherein the patient is afflicted with KS or HIV KLS.

27. The treatment method according to claim 26, where the compound is at least one antibody that binds to at least one protein selected from the group consisting of: CxCL11, CCR3, CCL1, CCR8, CCL2, and CCR2.

28. A system for treating a patient, comprising:
a human serum sampling handling device;
at least one protein probe that selectively binds at least protein selected from the group consisting of: IL-6, IL-13, sTNFRII, CCL1 (I-309), CCL2 (MCP-1), and CxCL11 (I-TAC);
a detector that monitors the at least one probe and records a signal from the probe that is proportional to the level of at least protein in the serum sample, where the at least one protein is selected from the group consisting of; IL-6, IL-13, sTNFRII, CCL1 (I-309), CCL2 (MCP-1), and CxCL11 (I-TAC) and wherein the detector produces a signal that is proportional to the level of the at least one protein selected from the group consisting of; IL-6, IL-13, sTNFRII, CCL1 (I-309), CCL2 (MCP-1), and CxCL11 (I-TAC);
a central processing unit, wherein the central processing unit is configured to receive the signal from the detector and to access a matrix of values for the levels of at least one protein selected from the group consigning of IL-6, IL-13, sTNFRII, CCL1 (I-309), CCL2 (MCP-1), and CxCL11 (I-TAC), determined in patients that are both diagnosed with and diagnosed without Kawasaki Disease or KLS, wherein the central processing unit compares the levels of the at least one protein quantified in the serum sample and in the matrix and produces an output; and
a user interface that receives the output from the central processing unit and displays a readable image of the comparison of the levels of protein in the serum sample and the values in the matrix.

29. The system according to claim 28, wherein the sample handling device is selected from the group consisting of: single well plates, multi-well plates, tubes, vials, syringe bodies, and aspirators.

30. The system according to claim 28, wherein the protein probe includes at least one antibody that has been raised to at least of the proteins selected from the group consisting of: IL-6, IL-13, sTNFRII, CCL1 (I-309), CCL2 (MCP-1), and CxCL11 (I-TAC).

31. The system according to claim 28, wherein the protein probe comprises Multiplex ELISA.

32. The system according to claim 28, wherein the detector records a change in at least one signal generated by the interaction between proteins in the serum sample the protein probe, wherein the signal is selected from the group consisting of: luminescence, fluorescence, conductivity, chemi luminescence, and radiation.

33. The system according to claim 28, wherein the central processing unit is a digital computer.

34. The system according to claim 28, wherein the user interface is selected from the group consisting of: a monitor and a printer.

35. The system according to claim 28, wherein the central processing unit is configured to produce an output that includes flagging a difference in the level of at least one protein selected from the group consisting of IL-6, IL-13, sTNFRII, CCL1 (I-309), CCL2 (MCP-1), and CxCL11 (I-TAC), of at least 1 standard deviation measured between said proteins in the serum sample and the values for said proteins stored in the matrix for individuals that have not been diagnosed with Kawaski disease or KLS.

36. The system according to claim 28, wherein the central processing unit is configured to produce an output that includes flagging a difference in the level of at least one protein selected from the group consisting of: IL-6, IL-13, sTNFRII, CCL1 (I-309), CCL2 (MCP-1), and CxCL11 (I-TAC), of at least 2 standard deviations measured between said proteins in the serum sample and the values for said proteins stored in the matrix for individuals that have not been diagnosed with Kawaski disease or KLS.

37. The system according to claim 28, wherein the central processing unit is further configured to receive at least one additional input, wherein the at least one additional input includes the presence of a least one gross physical manifestation of Kawaski disease or KLS in the patient from which the serum sample was obtained.

38. The system according to claim 37, wherein the central processing unit is further configured to produce an output that notes the existence of the existence of the at least one gross physical manifestation of Kawaski disease or KLS and transmits the note to the user interface.
